(19) United States
(12) Patent Application Publication
(10) Pub. No.: US 2020/0147294 A1
(43) Pub. Date: May 14, 2020

(54) SUCTION IRRIGATOR

(71) Applicant: TSALYS Medical Technologies Ltd, Shenzhen (CN)

(72) Inventor: Lee David Edwards, Wu Kai Sha (CN)

(21) Appl. No.: 16/744,094

(22) Filed: Jan. 15, 2020

Related U.S. Application Data
(63) Continuation of application No. PCT/CN2017/094649, filed on Jul. 27, 2017.

(51) Int. Cl. A61M 3/02; A61M 1/00

(52) U.S. Cl. CPC A61M 3/0283; A61M 1/0064; A61M 2205/3334; A61M 3/0254; A61M 3/022; A61M 1/0084

(57) ABSTRACT

Disclosed is a suction irrigator, including a tri-lumen tube which includes a normal-pressure irrigation lumen, a high-pressure irrigation lumen and a suction lumen, a manifold connected to the tri-lumen tube, two flexible tubes, a control unit and a dual-drainage tube connected to the two flexible tubes, where the cross-sectional area of the suction lumen is larger than that of the normal-pressure irrigation lumen, and the cross-sectional area of the normal-pressure irrigation lumen is larger than that of the high-pressure irrigation lumen; and the normal-pressure irrigation lumen and the high-pressure irrigation lumen are connected to one of the flexible tubes and the suction lumen is connected to the other flexible tube by means of the manifold; and flow rates of gas or liquid in the two flexible tubes are controlled by the control unit. The suction irrigator has a good irrigation effect and is convenient to use.

### CROSS REFERENCE TO RELATED APPLICATIONS

The present application is a Continuation Application of PCT Application No. PCT/CN2017/094649 filed on Jul. 27, 2017. The contents of PCT/CN2017/094649 are all hereby incorporated by reference.

### BACKGROUND OF THE INVENTION

### 1. Field of the Invention

The present application relates to a surgical instrument, in particular to a suction irrigator.

### 2. Description of the Prior Art

Suction irrigators are important tools used in laparoscopic surgeries and open surgeries. They supply irrigation fluid to the surgical site to clear views of bleeding sites and suction via vacuum to remove blood and other fluids and tissue debris from the surgical site. They are particularly useful for identification and precise location of any bleeding sites prior to using RF energy or electro-cautery for cutting. The suction irrigators are typically used through a 5 mm or 10 mm trocar access port or trocar used in conjunction with CO₂ insufflators, to maintain insufflation of the body cavity and to create viable operative space, and to pass instruments into the surgical cavity. Traditional suction irrigators comprise a single lumen tube (most commonly with an outer diameter of 5 mm, but sometimes 10 mm) and a valve or multiple valves often characterized as “Trumpet” valves, that can connect the tube to either suction or irrigation. The devices are typically made from plastic if disposable, or stainless steel if reusable, with the latter commonly dismantled, cleaned and sterilized between patients and surgical lists. The valves need to be dismantled for effective cleaning and decontamination. As such, they are difficult to clean because of valve and tube geometry. Therefore, disposable devices are now commonly used.

The suction irrigators use mostly a single-lumen tube to supply either irrigation or suction through a common lumen, and sharing one lumen for both suction and irrigation also results in the “dirty” liquid (contaminated with opaque fluids or solid matters such as blood and blood clots) previously removed being reintroduced into the patient, which makes detection of bleeding sites more difficult. It also commonly results in potentially contaminated fluid undesirably residing within the tube and then returning to the surgical site due to inadequate clearing of fluids from the tube. This is commonly known as “dripping” of the surgical site, and creates additional risk of re-infecting the surgical site. There are suction irrigators via two different lumens in the prior art, one for suction and one for irrigation. When used for irrigation, the flow of liquid is most often of low flow rate and flows slowly from the tube, therefore, existing technologies provide additional pressure to the irrigation supply either from a disposable pump or a pressurized vessel (e.g. nitrogen pressurized), adding additional cost and risk to the use of the device. While this gives better washing, the high flow rates consume a lot of irrigation fluid, requiring frequent suction of the excess fluid. Pressures must also be kept relatively low to control the total energy in the fluid stream, as excessive pressure causes unnecessary hurts to the surgical site. Therefore, there is no suction irrigator which has a good irrigation effect and is convenient to use in the prior art.

### SUMMARY OF THE INVENTION

In order to solve the problems of poor irrigation effect and inconvenient use of suction irrigators in the prior art, the present application provides a suction irrigator.

In order to solve the problems, the technical solution adopted by the present application is as follows.

A suction irrigator comprises: a tri-lumen tube, a manifold, two flexible tubes, a control unit and a dual-drainage tube, where the tri-lumen tube comprises a normal-pressure irrigation lumen, a high-pressure irrigation lumen and a suction lumen, the cross-sectional area of the suction lumen is larger than that of the normal-pressure irrigation lumen, and the cross-sectional area of the normal-pressure irrigation lumen is larger than that of the high-pressure irrigation lumen; the tri-lumen tube is connected to the manifold, the normal-pressure irrigation lumen and the high-pressure irrigation lumen of the tri-lumen tube are connected to the irrigation flexible tube by means of the manifold, and the suction lumen of the tri-lumen tube is connected to the suction flexible tube by means of the manifold; and flow rates of gas or liquid in the two flexible tubes are controlled by the control unit, and the two flexible tubes are connected to the dual-drainage tube.

Preferably, the control unit comprises an irrigation controller, a suction controller and a controllable knob.

Preferably, flow rates of gas or liquid in the normal-pressure irrigation lumen and the high-pressure irrigation lumen are controlled by the irrigation controller.

Preferably, a flow rate of gas or liquid in the suction lumen is controlled by the controllable knob and/or the suction controller.

Preferably, the suction irrigator further comprises a compression unit comprising a flat spring, a compression button and an M-shaped base, where the flat spring is connected to the irrigation controller and the suction controller, and when the irrigation controller and/or the suction controller apply/applies no pressure to the flat spring, the flat spring forces the compression button to press the flexible tubes to the M-shaped base; and when the irrigation controller and/or the suction controller apply/applies pressure to the compression button, the compression button is lifted away from the M-shaped base to allow the flexible tubes to be fully or partially opened.

Preferably, gas and liquid flows in the suction lumen are accurately and continuously controlled by the controllable knob.

Preferably, the suction lumen is linear.

Preferably, the suction irrigator further comprises an irrigation pump or an elevated fluid bag serving as a source of high-pressure irrigation fluid.

The present application has the beneficial effects that: a suction irrigator with a tri-lumen tube is provided, and the tri-lumen tube comprises a normal-pressure irrigation lumen, a high-pressure irrigation lumen and a suction lumen; during an operation, the suction irrigator can provide both normal-pressure irrigation and high-pressure irrigation as required; moreover, the suction lumen has a large aperture and is not easily blocked; and aspirated and irrigated gas or liquid flows are completely independent, so that the suction irrigator has a good irrigation effect and is convenient to use.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic view showing an external structure of a suction irrigator of Embodiment 1 of the present application.

FIG. 2 is a schematic view showing an internal structure of the suction irrigator of Embodiment 1 of the present application.

FIG. 3 is a schematic view showing an internal structure of another suction irrigator of Embodiment 1 of the present application.

FIG. 4 is a schematic cross-sectional view of a tri-lumen tube of Embodiment 1 of the present application.

FIG. 5 is a schematic view of a controllable knob of Embodiment 1 of the present application.

FIG. 6 is a schematic view showing an operation state of the controllable knob of Embodiment 1 of the present application.

FIG. 7 is a schematic view showing an operation state of another controllable knob of Embodiment 1 of the present application.

In the figures, 1—tri-lumen tube, 2—manifold, 3—suction controller, 4—irrigation controller, 5—suction flexible tube, 6—irrigation flexible tube, 7—normal-pressure irrigation lumen, 8—high-pressure irrigation lumen, 9—suction lumen, 10—flat spring, 11—M—shaped base, 12—controllable knob, 13—compression button, and 14—rotation gradual change mechanism.

### DETAILED DESCRIPTION

The present application will be described in detail below by way of specific embodiments with reference to the accompanying drawings so as to provide a better understanding of the present application, but the following embodiments do not limit the scope of the present application. In addition, it is to be understood that the drawings, which are provided in the following embodiments, illustrate the basic concept of the present application by way of illustration only, the drawings show only the components related to the present application and are not drawn in terms of the number, shapes and sizes of the components in actual practice, the shapes, number and sizes of the components in actual practice may be varied at will, and the layout of the components may be more complex.

### Embodiment 1

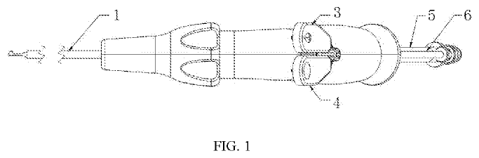

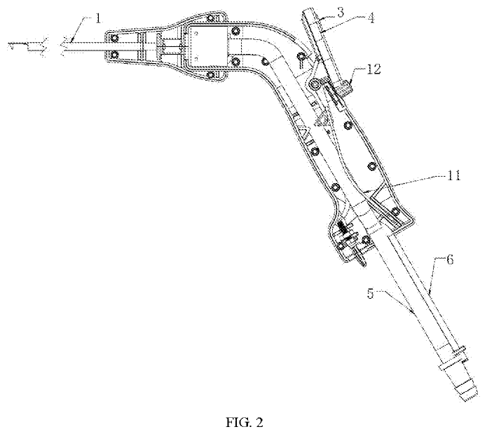

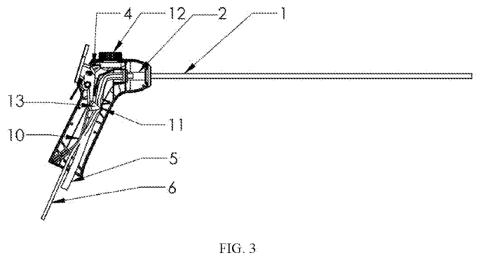

As shown in FIGS. 1, 2 and 3, a suction irrigator comprises: a tri-lumen tube 1, a manifold 2, two flexible tubes 5, 6, a control unit and a dual-drainage tube which are sequentially connected.

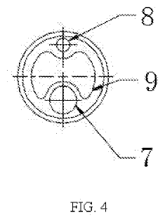

As shown in FIG. 4, the tri-lumen tube comprises a normal-pressure irrigation lumen 7, a high-pressure irrigation lumen 8 and a suction lumen 9, the cross-sectional area of the suction lumen 9 is larger than that of the normal-pressure irrigation lumen 7, and the cross-sectional area of the normal-pressure irrigation lumen 7 is larger than that of the high-pressure irrigation lumen 8; the tri-lumen tube 1 is connected to the manifold 2, and the normal-pressure irrigation lumen 7 and the high-pressure irrigation lumen 8 of the tri-lumen tube 1 are connected to the irrigation flexible tube 6 and the suction lumen 9 of the tri-lumen tube 1 is connected to the suction flexible tube 5 by means of the manifold 2; and flow rates of gas or liquid in the two flexible tubes are controlled by the control unit, and the two flexible tubes are connected to the dual-drainage tube. During an operation, the suction irrigator can provide both normal-pressure irrigation and high-pressure irrigation as required. Moreover, the suction lumen 9 is linear, and the suction lumen 9 has a large aperture and is not easily blocked. Aspirated and irrigated gas or liquid flows are completely independent, so that the suction irrigator has a good irrigation effect and is convenient to use.

In an alternative embodiment of the present application, the control unit comprises an irrigation controller 4, a suction controller 3 and a controllable knob 12, and gas and liquid flows in the suction lumen are accurately and continuously controlled by the controllable knob 12.

In a further alternative embodiment of the present application, flow rates of gas or liquid in the normal-pressure irrigation lumen 7 and the high-pressure irrigation lumen 8 are controlled by the irrigation controller; and a flow rate of gas or liquid in the suction lumen 9 is controlled by the controllable knob and/or the suction controller.

In yet another alternative embodiment of the present application, the suction irrigator further comprises compression units for compression on the flexible tubes, and each flexible tube is provided with one compression unit. The compression unit comprises a flat spring 10, a compression button 13 and an M-shaped base 11, where the flat spring 10 is connected to the irrigation controller 4 and the suction controller 3, and when the irrigation controller 4 and/or the suction controller 3 apply/applies no pressure to the flat spring 10, the flat spring 10 forces the compression button 13 to press the flexible tubes to the M-shaped base 11; and when the irrigation controller 4 and/or the suction controller 3 apply/applies pressure to the compression button 13, the compression button 13 is lifted away from the M-shaped base 11, allowing gas or liquid to flow through the flexible tubes.

The part of the compression button 13 compressing against the M-shaped base 11 is wedged, so that a corner of the compression button 13 can be just pressed into a gap of the M-shaped base, and the flow rates of gas or liquid in the flexible tubes can be effectively controlled and prevented from backflow.

In a fourth alternative embodiment of the present application, the suction irrigator further comprises an irrigation pump or an elevated fluid bag serving as a source of high-pressure irrigation fluid.

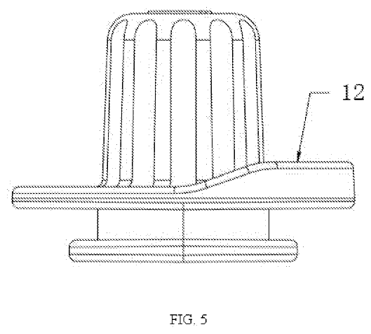

As shown in FIG. 5, the controllable knob 12 may be used to control the flow of gas in the suction lumen when smoke control is required during an operation. The controllable knob 12 allows precise control, and when rotated clockwise or counterclockwise, the controllable knob 12 adjusts the degree of compression on the flexible tube connected to the suction lumen as the ascending thickness of the rotation gradual change mechanism 14. This degree of compression can be controlled and maintained after the adjustment with the button without continuous pressing operation, so that the use is convenient.

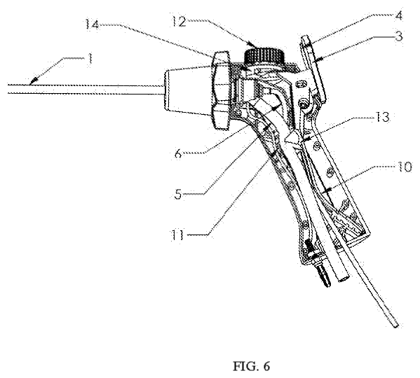

As shown in FIG. 6, the rotation gradual change mechanism 14 of the controllable knob 12 applies no pressure through the suction controller 3, and the flexible tubes 5 are completely depressed by the compression button 13 such that no gas and/or liquid flows through the flexible tubes 5.

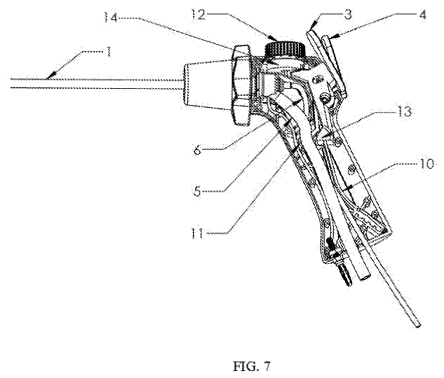

As shown in FIG. 7, the rotation gradual change mechanism 14 of the controllable knob 12 applies pressure through the suction controller 3, and the pressure applied to the flexible tubes 5 by the compression button 13 is reduced such that the partial or complete blocking of the flexible tubes 5 is released to allow gas and/or liquid to flow through the flexible tubes 5, thereby achieving accurate control of irrigation and suction.

The overall handle layout is a pistol grip design; this is more ergonomic in use, with better surgeon hand and arm positions than with the traditional “in line” valve or trumpet valve layout. The functions of suction and irrigation are controlled by the thumb of either the left or right hand to give improved control and feel.

The foregoing is a further detailed description of the present application with reference to specific preferred embodiments, and it is not to be construed that the specific embodiments of the present application are limited to these descriptions. For a person skilled in the art of the present application, several equivalent substitutions or obvious modifications can be made without departing from the concept of the present application, and same properties or uses should be considered to fall within the scope of protection of the present application.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A suction irrigator, comprising: a tri-lumen tube, a manifold, two flexible tubes, a control unit and a dual-drainage tube,
wherein the tri-lumen tube comprises a normal-pressure irrigation lumen, a high-pressure irrigation lumen and a suction lumen, the cross-sectional area of the suction lumen is larger than that of the normal-pressure irrigation lumen, and the cross-sectional area of the normal-pressure irrigation lumen is larger than that of the high-pressure irrigation lumen;
the tri-lumen tube is connected to the manifold, the normal-pressure irrigation lumen and the high-pressure irrigation lumen of the tri-lumen tube are connected to one of the flexible tubes by means of the manifold, and the suction lumen of the tri-lumen tube is connected to the other flexible tube by means of the manifold; and
flow rates of gas or liquid in the two flexible tubes are controlled by the control unit, and the two flexible tubes are connected to the dual-drainage tube.

2. The suction irrigator of claim 1, wherein the control unit comprises an irrigation controller, a suction controller and a controllable knob.

3. The suction irrigator of claim 2, wherein flow rates of gas or liquid in the normal-pressure irrigation lumen and the high-pressure irrigation lumen are controlled by the irrigation controller.

4. The suction irrigator of claim 2, wherein a flow rate of gas or liquid in the suction lumen is controlled by the controllable knob and/or the suction controller.

5. The suction irrigator of claim 2, further comprises: a compression unit comprising a flat spring, a compression button and an M-shaped base, wherein the flat spring is connected to the irrigation controller and the suction controller, and when the irrigation controller and/or the suction controller apply/applies no pressure to the flat spring, the flat spring forces the compression button to press the flexible tubes to the M-shaped base; and when the irrigation controller and/or the suction controller apply/applies pressure to the compression button, the compression button is lifted away from the M-shaped base to allow the flexible tubes to be fully or partially opened.

6. The suction irrigator of claim 2, wherein gas and liquid flows in the suction lumen are accurately and continuously controlled by the controllable knob.

7. The suction irrigator of claim 1, wherein the suction lumen is linear.

8. The suction irrigator of claim 2, wherein the suction lumen is linear.

9. The suction irrigator of claim 3, wherein the suction lumen is linear.

10. The suction irrigator of claim 4, wherein the suction lumen is linear.

11. The suction irrigator of claim 5, wherein the suction lumen is linear.

12. The suction irrigator of claim 6, wherein the suction lumen is linear.

13. The suction irrigator of claim 1, further comprises: an irrigation pump or an elevated fluid bag serving as a source of high-pressure irrigation fluid.

14. The suction irrigator of claim 2, further comprises: an irrigation pump or an elevated fluid bag serving as a source of high-pressure irrigation fluid.

15. The suction irrigator of claim 3, further comprises: an irrigation pump or an elevated fluid bag serving as a source of high-pressure irrigation fluid.

16. The suction irrigator of claim 4, further comprises: an irrigation pump or an elevated fluid bag serving as a source of high-pressure irrigation fluid.

17. The suction irrigator of claim 5, further comprises: an irrigation pump or an elevated fluid bag serving as a source of high-pressure irrigation fluid.

18. The suction irrigator of claim 6, further comprises: an irrigation pump or an elevated fluid bag serving as a source of high-pressure irrigation fluid.
